(19) United States
(12) Patent Application Publication
(10) Pub. No.: US 2011/0151010 A1
(43) Pub. Date: Jun. 23, 2011

(54) ANTI-SNORING TREATMENT COMPRISING POSITIVELY CHARGED MULTILAMELLAR MICROPARTICLES

(75) Inventors: ALEX DUGGAN, Epsom (GB); JEAN-LOIC BARATOUX, Courbevoie (FR)

(73) Assignee: Passion for Life Healthcare Limited

(21) Appl. No.: 13/037,923

(22) Filed: Mar. 1, 2011

Related U.S. Application Data
(62) Division of application No. 10/580,753, filed on Nov. 30, 2006.

(30) Foreign Application Priority Data
Nov. 29, 2003 (GB) 0327814.0
Jun. 12, 2004 (GB) 0413139.7

(51) Int. Cl. A61K 9/14; A61K 36/00; A61K 36/28; A61K 36/87; A61P 11/00

(52) U.S. Cl. 424/489; 424/778; 424/764; 424/766

(57) ABSTRACT

A composition for nasal or buccal application comprising a distribution of multilayer microparticles in an inactive base, at least one ingredient having activity on the mucosa of the nose/throat, being adsorbed within the layers of the microparticles so as to be progressively released over time in use.

The present application is a divisional application of U.S. patent application Ser. No. 10/580,753 filed Nov. 20, 2006, which in turn is the U.S. national phase of International Patent Application No. PCT/GB2004/005010, filed Nov. 29, 2004, which claims priority to GB 0413139.7, filed Jun. 12, 2004, and GB 0327814.0, filed Nov. 29, 2003, all of which are incorporated by reference.

### BACKGROUND OF THE INVENTION

The invention relates to a composition for delivery of ingredients active in situ to a site on the mucous membranes of the nose or throat of a human or animal subject. The invention also relates to a method of preparation of such a composition, and to such a composition within a suitable delivery system. The invention in particular relates to an aerosolisable composition for use as a nasal or throat spray, to a method of preparation of such an aerosolisable composition, and to such a composition as an aerosolisable spray within a suitable delivery system.

Various delivery systems are known by which an active ingredient, such as a medicament or the like, can be delivered to an active site on the mucous membranes of the nose or throat. These include active ingredients in solution or liquid suspension, for oral administration by swallowing, gargle or rinse; active ingredients within a soluble solid carrier such as a pastille, lozenge or the like; and active ingredients in solution or suspension in an aerosolisable form, for use in a nasal or throat spray. The present invention is particularly effective in the provision of preparation suitable for an aerosolisable delivery, and examples of nasal and throat sprays are given. However, it should be understood that the invention is not limited to aerosolisable delivery systems, and that any other delivery systems, for example, based on active ingredients in solution or suspension in a liquid, or in a solid soluble carrier form, could be envisaged.

The general principle of using a spray directed at the mucous membranes of the nose or throat to deliver an active ingredient thereto is well established. A spray can provide an effective way to deliver a controlled and effective dose of an active ingredient to a desired site in the nose or throat of a subject in particular to produce a desired physical or pharmacological effect. Such active ingredients might include for example decongestants, breath-fresheners and deodorisers, lubricants, antibacterial and antiseptic compositions, anti-histamines, anti-inflammatory compositions, analgesics, medicaments to treat to specific conditions associated with the mucous membrane in situ, and medicaments intended to be absorbed across the mucous membrane for active effect elsewhere.

In particular, throat sprays are known as a means to deliver a composition intended to reduce the impact of snoring in a subject. Such compositions are intended primarily to tackle the social effect of the snore. That is, they are intended particularly to attenuate the noise of the snore, and to reduce its impact, in particular as perceived by other parties, rather than to treat any underlying condition as such. Compositions are prepared comprising one or more lubricant active ingredients intended to keep the soft tissues and mucous membranes of the nose and pharynx moist and lubricated and thus reduce the noise associated with snoring, and in particular the noise associated with snoring which arises from the soft tissues of the throat.

Various known preparations exist for this purpose. These typically include a mixture of various active nature oils designed to be sprayed into the throat onto the mucous membranes at the back of the throat and thereby to provide for a lubrication and/or a moisturizing effect on the soft tissues of the throat. Alternative compositions also exist primarily directed at alleviating the noise attributable to nasal snoring and applied as a nasal spray onto the nasal membranes though these can be less effective, particularly in cases where the nasal snoring is attributable at least in part to nasal congestion. Compositions may include additional function other than that of keeping the mucous membrane moist, for example including active ingredients having decongestant properties.

Such compositions can be limited in effectiveness, particularly over time. It is inherent in the nature of the problem that they are intended to solve that sustained activity over a sustained period, and most preferably overnight is desirable. However in practice the effect of spraying lubricants on to the mucous membranes of the nose or throat is likely to be more short lived as the active ingredient is rapidly lost from the desired site through for example evaporation, the action of secreted nasal mucus and saliva etc.

To limit this problem a means to stabilise the active ingredient in situ on the mucous membrane is required, and this has led in some current spray compositions to the use of liposome technology to bind the active ingredients more effectively. Even so, there is still a tendency for the activity of such ingredients to decrease significantly with time, so that the sprays offer an effectiveness which in practice is very much less than the duration of a full night's sleep.

### BRIEF SUMMARY OF THE INVENTION

It is an object of the present invention to provide a composition in particular but not limited to an aerosolisable composition for nasal or buccal application which mitigates some or all of the above disadvantages of prior art systems.

It is a particular object of the present invention to provide a composition in particular but not limited to an aerosolisable composition relying on known and/or new active ingredients which serves to stabilise the active ingredients in situ on the mucous membranes of the nose or throat for a sustained period of time.

It is a particular object of the present invention to provide a composition in particular but not limited to an aerosolisable composition relying on known and/or new active ingredients which ensures that activity of the active ingredient is sustained for an increased period of time, and in particular retains reasonable activity levels overnight.

It is a particular further object of the invention to provide a delivery system for such a composition, in particular but not limited to a nasal or throat spray delivery system for an aerosolisable composition.

Thus according to the invention in its broadest aspect there is provided a composition for nasal or buccal application comprising a distribution of multilayer microparticles in a base, at least one ingredient having activity on the mucosa of the nose/throat, being adsorbed within the layers of the microparticles so as to be progressively released over time in use.

### BRIEF DESCRIPTION OF THE DRAWINGS

FIG. 1 illustrates a suitable pump spray dispenser suitable for use with a composition in accordance with the invention intended for use as a throat spray.

FIG. 2 is a SPHERULITE™ microparticle suitable for use with a composition in accordance with the invention.

### DETAILED DESCRIPTION OF THE INVENTION

In accordance with the invention, multilayer microparticles are distributed within a suitable base, for example a fluid base which serves as the means for delivery of the microparticles to the mucosa of the nose/throat. The base may be inactive, or may have a complementary or other activity. The composition of the invention is particularly suited to use as an aerosolisable composition for nasal or buccal application by means of a spray, mousse or drench. In this case the composition comprises a suspension of microporous microparticles in a suitable liquid base. A spray, drench or mousse is ideally suited to applying a controlled dose to the required site, for example on the membranes at the back of the throat or nasal membranes, where the active ingredient is then retained for slow release.

In the alternative, the composition comprises a suspension in a suitable liquid base for direct oral or nasal administration, for example by oral ingestion, gargle, rinse or the like. In a further alternative the composition comprises a distribution of multilayer microparticles in a soluble solid or gel base, in particular for oral administration, for example as a pastille, lozenge or the like, the base material being such as to dissolve within the mouth and liberate the microparticles to allow them to find their site on the mucous membranes of the throat.

The multilayer microparticles are selected to exhibit good adhesion to the mucous membranes of the nose and/or throat, and are small enough to be aerosolisable as a spray. The layers are structured to give slow release of the active ingredient over the desired time period, so that the spray gives sustained activity over time, for example providing for measurable activity (e.g., at least 50% of initial base line activity level) for a sustained period of four or more hours, and ideally of for example 6 to 12 hours, to give overnight effectiveness. The microparticles are sized and shaped in accordance with the required delivery means, for example to form an effectively aerosolisable fluid phase in suspension as a composition in accordance with the invention. The microparticles in particular comprise generally spherical particles or microspheres. Particle sizes in the range 0.1 to 50 μm, and for example 1 to 20 μm are likely to be preferred. In the preferred embodiment particle levels of 10-25% within the composition are likely to be suitable to optimise effect whilst obtaining an aerosolisable fluid phase in suspension.

The microparticles are adapted to facilitate slow release of the active ingredients over time, and are found inherently to show good adhesion to the mucous membranes of the nose and/or throat. The net result of this is that the active ingredients are stabilised in situ on the mucous membranes, and that the active ingredients are then released steadily at the site were they are required. Loss of activity over time is significantly reduced compared with conventional sprays relying for example on liposome technology, and it becomes possible to maintain reasonable levels of activity over the sort of time scale necessary to be effective overnight, and for example to assist in providing a relatively less disturbed night's sleep.

Whilst the invention is not limited by any particular theory of operation, it is considered that the adhesive effect arises in particular in that the microparticles generally comprise polar structures with a positive surface charge. Adhesion to the mucosa of the nose or throat is particularly effective as the tissue in the nose or throat tends to be negatively charged.

The particles are multilamellar and can present a mixture of hydrophilic and lipophilic surfaces depending on the chosen active ingredients. These surfaces serve to bind the active ingredients within the layers and facilitate slow release as each layer is successively destroyed by enzymes in the nose or mouth to release the active ingredient within the layer. In particular the microparticles comprise multiple layered structures formulated with one or more of and preferably examples of all of: surfactant layers (comprising any type of surfactant such an anionic, non-anionic, cationic, phospholipids and the like such as sucroesters and guar hydroxypropltrimonium chloride); solvents or polar media such as water, glycerol, PEG, sorbitol, glycol; and active encapsulated materials comprising hydrophilic materials such as alcohols or ethoxylated alcohols, carboxylic acids or salt of a fatty acid, quaternary ammonium derivatives, sulphonates or sulphates and the like, which might for example include vitamins (B, C), flavondïdes, 18-beta glycyrrhetinic acid and derivate, glycerol, plant extract, hydrophilic preservative, cellulose polymer, hyaluronic acid and derivate, alpha-hydroxide acid, etc, in the polar layers; and hydrophobic or liophilic materials such as aliphatic and aromatic hydrocarbons, optionally halogenated, higher alcohols, ketones and the like, for example including Vitamins (A, E, D), carotenoïdes, vegetable oils, essential oils, phytosterols, lipophilic preservatives, menthol, linalool, eucalyptol, etc, in the surfactant layers.

The positively charged polymer is preferably selected from in oral strips: Pectin; cellulose; sodium hyaluronate; guar hydroxypropyltrimonium chloride; polysorbate 60.

In throat spray:

polysorbate 60; cellulose; xanthan gum; sodium hyaluronate; guar hydroxy-propyltrimonium chloride, chitosan or quaternary ammonium.

In nasal spray:

polysorbate 60; cellulose; xanthan gum; guar hydroxypropyltrimonium chloride, obit/manor quaternary ammonium.

Preferably the composition comprises:

### Solvent 30-60%,

### Humectant 8-14%,

### Texturant 0-2%,

### Preservative 0-2%,

Acidity regulator 0-1%,
And microparticles as hereinbefore defined 10-50%.

In the preferred embodiment where the microparticles are microspheres such a multiple layered structure in particular comprises substantially concentric spheroidal surfaces. These multiple layered structures are particularly suited to the controlled release of active ingredients adsorbed within the microparticles over a controlled period of time.

The microparticles thus preferably comprise multi-lamellar structures of surfactant layers, which are able to encapsulate active ingredients to a very high degree for protection and controlled slow release. The surfaces of the microparticles are such as to be adapted to enhance adhesion to human skin, and hence to fix the particles in position on the mucous membranes of the nose and/or throat as the active ingredients are progressively released. In a particularly effective mechanism, slowly, over time, the layers of the microsphere are dispersed successively by the mucosal enzymes and fluids, releasing the active ingredients as they do so. Slow release of active ingredients in situ is thereby effected.

Suitable compositions include 30 to 50% surfactant, 30 to 90% polar medium, and 1 to 50% active encapsulated agent, comprising hydrophilic and hydrophobic agents as appropriate. Encapsulation ratio is 0 to 45% for hydrophilic agents and 0 to 20% for hydrophobic agents.

Preferably microparticles comprise 10-70% solvent, 5-15% surfactant, 0.1-10% humectant, 0.1-3% lubricant, 0-2% aroma/flavour, 0-1% antioxidant, 0.01-1% preservative.

Microparticles such as are above described have been extensively developed for cosmetic application. They are found to give goods skin adhesion to stabilise colour, gloss etc in the desired position, increase effect life of the cosmetic product in situ etc. They have not hitherto been described in relation to the controlled binding for slow release of physically active ingredients at the active site on the mucous membrane of the nose or throat of the human, non-human mammal or other animal subject in the manner of the present invention. However, in accordance with the present invention, they are found to be surprisingly effective for such an application, both because the microparticles bind effectively to the membranes to ensure good delivery in situ, and because they lend themselves ideally to the controlled slow release of the microencapsulated active ingredients.

Microparticles are commercially available, e.g., SPHERULITE™ (Capsulis SA). The particle size is 0.1 to 10 μm.

Adsorbed within of the microparticles on or between the surfaces of the multiple layers thereof are one or more different active ingredients having activity on the mucous membranes of the nose and/or throat as the case may be. In a preferred embodiment, for application to limit the physical effects of snoring, these active ingredients include at least one active ingredient to lubricate and/or moisturise the mucous membrane, to ease breathing and reduce snoring. However the invention is not limited to active ingredients with this activity, but could include active ingredients with other activities, for example physical (moisturising, lubricating, cooling etc) or pharmacological (for example decongestant, anti-histamine, anti-bacterial, anti-inflammatory, analgesic etc).

In the preferred embodiment the active ingredient comprises one or more lubricant/moisturisers to lubricate and/or moisturise the nasal and/or throat membranes as the case may be. In particular, the composition is for nasal application for example as a nasal spray composition and the active ingredient comprises one or more lubricant/moisturiser to lubricate and/or moisturise the nasal membranes. Alternatively, the composition is for buccal application for example as a throat spray composition and the active ingredient comprises one or more lubricant/moisturiser to lubricate and/or moisturise the throat membranes.

Natural oils and the like are especially preferred active ingredients. For example, the active ingredient may comprise a mixture of lubricating and/or moisturising oils selected from the group comprising: hyaluronic acid or sodium hyaluronate, glycerol, Calendula officinalis flower extract or glycerin extract, guar hydroxypropyltrimonium chloride, xanthan gum, cellulose gum, sodium chloride, Olivum (olive oil), Helianthus annus (sunflower oil), Prunus dulcis (sweet almond oil), Sesamum indicum (sesame oil), Aloe vera, Aloe barbadensis, Euphorbium officinarum, oxymetazoline hydrochloride, lactoperoxidase and combinations thereof.

Additionally or alternatively the composition preferably comprises as an active ingredient at least one decongestant, being an ingredient having a chemical or pharmacological or other effect of reducing airway congestion and/or limiting further airway mucus production. In particular, the additional ingredient comprises a nasal decongestant selected to clear and/or limit the further production of nasal mucus. The active ingredient may be a natural oil, a pharmacologically active synthetic preparation, or combination.

Suitable examples include: hyaluronic acid, Calendula officinalis flower extract or glycerin extract, Thymus vulgaris, menthyl lactate, Mentha piperita (or any other mint/peppermint derivative or extract), Lavendula augustifolia (or any other lavender derivative or extract), phenylephrine hydrochloride, pseudephedrine, ascorbic acid (vitamin C), acerola, Rumex crispus (yellow dock), Eucalyptus globulus (eucalyptus oil), levmetamfetamine, oxymetazoline hydrochloride, propylhexedrine, xylometazoline hydrochloride, zincum gluconicum, menthol, eugenol, cineol, rosemary oil (Rosmarinus), summer savory oil (Satureia hortensis), wild thyme oil (Thymus serpyllum), firtree oil, Lavendula vera oil, geranium oil, cinnamon oil, hawthorn extract (Crataegus oxyacantha), rose hips extract (Rosa canina), cypress oil (Cupressus sempervirens), grapeseed oil and combinations thereof.

In an embodiment, the composition is adapted for nasal application, and incorporates the nasal decongestant as above described together with at least one lubricant and/or moisturiser. This is particularly effective. Systems which rely on lubrication and/or moisturising alone will be entirely ineffective against nasal snoring where a subject has an infectious, irritated or allergic breathing congestion, and such infectious, irritated or allergic breathing congestion is likely to exaggerate the undesirable effects of nasal snoring. For the same reason, a composition adapted for nasal application may also include an active ingredient with anti-histaminic action.

The microparticles fix the active ingredients adsorbed inside each shaped layer in position on the membranes of the nose or throat of the user, protect the active ingredients and slowly release them in situ, and might also assist in providing a desired lubricating effect.

The composition comprises a dispersion of multilamellar microparticles as above described having active ingredients encapsulated on the surface layers thereof and dispersed within a liquid base so as to be aerosolisable for application.

The liquid base may be any suitable base and is preferably aqueous, for example comprising a saline or otherwise generally isotonic solution.

Further active or inactive ingredients might be provided either encapsulated within the microparticles or separately in suspension or solution within the liquid base for various purposes. For example additional active ingredients might include further ingredients having any further desired physical or pharmacological activity on the mucous membranes of the nose and/or throat, including without limitation decongestants, breath-fresheners and deodorisers, lubricants, antibacterial and antiseptic compositions, anti-histamines, anti-inflammatory compositions, analgesics, and other medicaments and non-medicaments. Other ingredients might further be added in suspension or solution for example to stabilise or preserve the liquid base, balance the pH of the liquid base, bring the liquid base to closer approximation to isotonic concentration etc. Formulating agents may be present together with active ingredients as hereinbefore defined and may be selected from excipients, carriers, supports, binders, diluents, fillers, quick release agents, adhesion promoters, stabilisers, antioxidants, initiators, accelerators, buffers, hardeners, and the like.

In accordance with the further aspect of the invention, a method for the preparation of a composition for the controlled delivery of an active ingredient over time in situ at the mucous membranes of the nose or throat of a human, non-human mammal or other animal comprises the steps of:

microencapsulating at least one ingredient having activity on the mucosa of the nose/throat within or on the layer surfaces of a multilayer microparticle; and

distributing the ingredient within a suitable inactive base material serving as a means to deliver the microspheres to the active site for example suspending in a liquid base or distributing in a soluble solid base.

Methods of microencapsulation are known in the art, in particular from WO 93/19735, and WO 95/18601, the contents of which are incorporated herein by reference.

It is first necessary to manufacture the SPHERULITES™. The method of WO 95/18601 is generally followed initially, in that a mixture is formed of suitable binding materials, of an aqueous solvent phase, and of a surfactant. In accordance with the example compositions of the invention, this will typically involve the mixing of vegetable oils with surfactants and the glycerine-water phase.

The mixing process takes under shear, and the selection of an appropriate shear rate is of importance in eventual SPHERULITE™ formation. The process initially forms a homogenous lamellar liquid crystal phase as described in WO 95/18601, but subject to suitable control of the shearing conditions, the layers are formed into SPHERULITES™ as required by the invention.

Other ingredients are selected for the properties set out above, for example as surfactants, lubricants and the like. Example compositions are set out below. For example surfactants might be polysorbate 60, sorbitan stearate, and guar hydroxypropyltrimonium chloride, vegetable source. Polysorbate 60 and sorbitan stearate also have a lubricant function. Guar hydroxypropyltrimonium is a cationic surfactant, and serves to give a positive charge to the SPHERULITE™. This is believed to enable the SPHERULITES™ to be absorbed and remain on the mucus membrane, and to facilitate controlled release over time.

The SPHERULITES™ are formed as small multi lamellar vesicles with an onion-like structure which is very stable, flexible and solid. In situ on the mucus membranes, the SPHERULITES™ are progressively broken down by skin or mucus enzymes, layer by layer, so that the encapsulated ingredients are slowly released, and are able to lubricate and provide other activity over a sustained period on the surface onto which they are sprayed.

The second part of the manufacturing process is the incorporation of the SPHERULITES™ into a suitable base, for example dispersed within a liquid to form a spray, drench or mousse or the like. In an embodiment, the base comprises a dispersion of microspheres in an aqueous medium, comprising a purified water solvent with humectant, texturants, and optionally other stabilizing ingredients such as preservatives, acidity regulator and the like. For example, a gel-like medium is formed by the addition of texturants such as xanthan gum, microcrystalline cellulose, to a water/glycerine (humectant phase) at elevated temperature. SPHERULITES™ are introduced into the resultant solution at low temperature, and fully dispersed. Other stabilizing ingredients may be added. The result is a very stable liquid.

For use, the liquid is applied to the mucosa by a suitable delivery system, for example being sprayed to the back of the throat. The texturants, such as microcrystalline cellulose, limit flow of the liquid, tending to cause it to adhere in the initial phase. The positive charge on the SPHERULITES™ then cause the SPHERULITES™ themselves to adhere more strongly to the mucous membranes. The structure of each SPHERULITE™ is then broken, layer by layer, by enzymes in situ, producing a slow release of the encapsulated active ingredients.

The composition in accordance with the invention is preferably provided for use as a spray, and in particular as a nasal or throat spray. In particular the method comprises preparing a spray composition by preparing a suspension of a plurality of such particles in a liquid base and filling a spray dispenser of suitable design with the suspension, and in particular a nasal spray dispenser, comprising a base reservoir container containing a composition as hereinbefore described and a spray delivery system for example comprising a pump spray, the reservoir being fluidly connected to the spray delivery system, and the spray delivery system being adapted to draw, aerosolise and deliver a controlled dose from the reservoir to a subject in use.

In particular the spray delivery system might comprise a dose fluid reservoir to measure and dispense a predetermined dose of spray from the base reservoir in the container. Suitable spray technology will be familiar and is not specifically pertinent to the invention.

Alternatively, the method comprises preparing a liquid suspension of microparticles for direct oral administration, for example by ingestion, gargle or rinse. Further alternatively, the method comprises preparing a composition for oral administration distributed within a soluble solid or gel base, for example as a lozenge or pastille.

In a particular preferred embodiment the method comprises the specific steps of: preparing a suspension of a plurality of such particles in a liquid base; forming an aerosol spray from the said suspension.

In a further aspect of the invention, there is provided a method of delivering an active ingredient to the nose or throat of a human, non-human mammal or other animal subject for controlled release over time in situ at the mucous membranes of the subject comprises the steps of:

administering the composition to the subject in such manner that the microparticles are directed towards a desired site on the mucus membrane of the subject.

The active ingredient may have a therapeutic effect, for example exhibiting pharmacological or other physiological activity, or may have a non-therapeutic effect, for example in the reduction of the social effects of snoring and the like. Thus in one alternative, the method applies an active ingredient which is directly physiologically or pharmacologically active to treat a specified medical condition and therefore comprises a method of medical treatment. In another alternative the method comprises the application of an active ingredient which has a physical non-medical activity not specifically being a method of medical or therapeutic treatment. In a particular example of the latter, an active ingredient comprises a lubricant and/or moisturiser to lubricate or moisturise the mucous membranes of the nose or throat of a user to minimise the effects of snoring. The method thus serves not to treat any underlying condition which might be contributing to the snoring as such, but rather to attenuate the noise produced thereby and so provide relief to third parties from the anti-social effects of the noise associated therewith.

In a further aspect of the invention there is provided the use of the composition of the invention or an active microparticle as hereinbefore defined in progressive delivery of an active ingredient as hereinbefore defined.

In a further aspect of the invention there is provided a novel active microparticle as hereinbefore defined. Preferably a novel active microparticle comprises active ingredients effective in the reduction of snoring or apnoea.

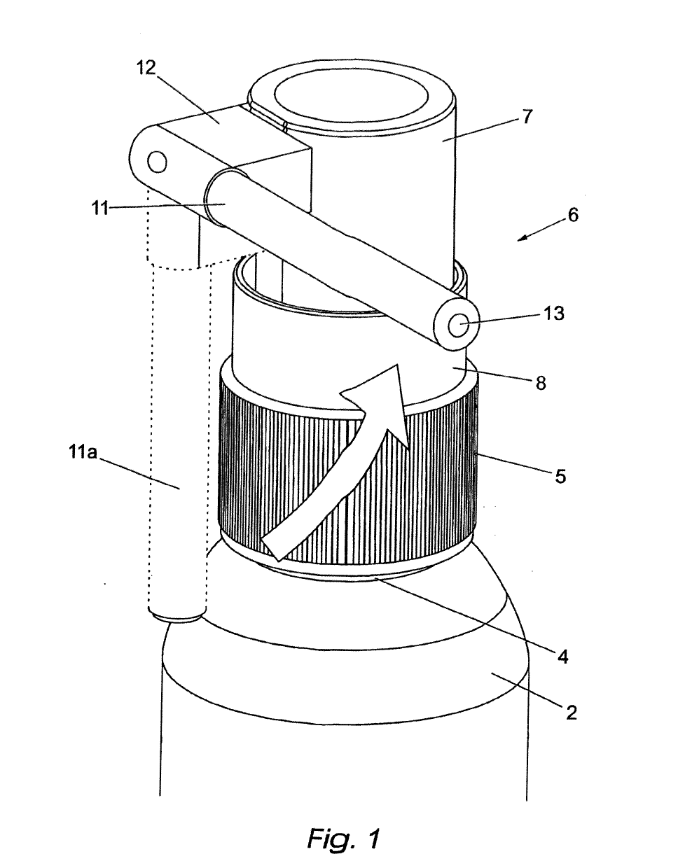

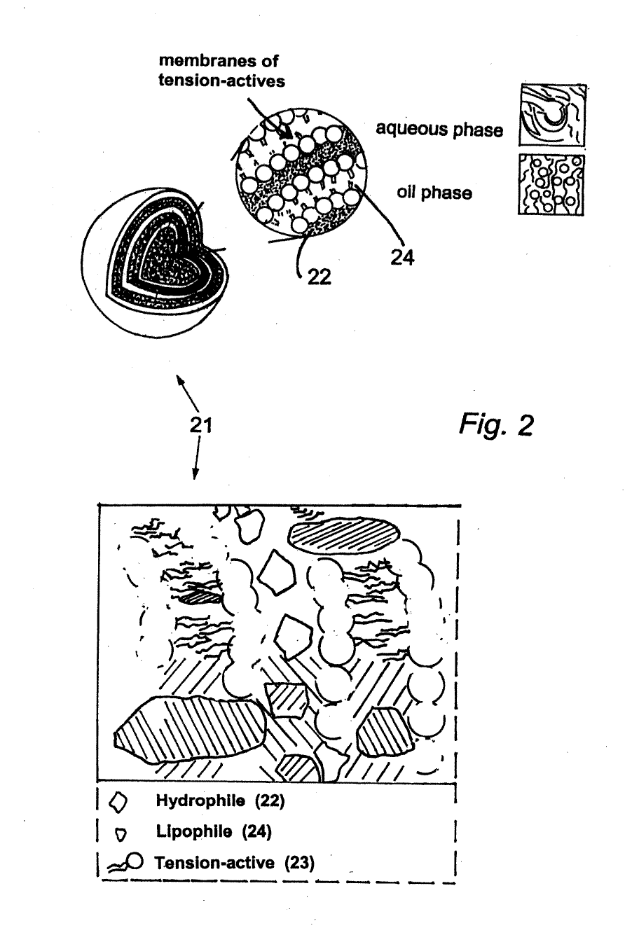

By way of example only, FIG. 1 illustrates a suitable pump spray dispenser suitable for use with a composition in accordance with the invention intended for use as a throat spray. FIG. 2 is a spherulite microparticle suitable for use with a composition in accordance with the invention.

In FIG. 1, the composition, comprising an aqueous suspension of multilamellar microparticles incorporating active ingredients as above described, is stored in a hygienic plastic or other bottle 2. The bottle has a screw threaded neck 4 onto which a pump dispenser unit 6 may be attached via a threaded portion 5. The thread may provide for the unit to be unscrewed for refilling or may lock. Other connections could be substituted.

The pump dispenser unit 6 comprises a chamber portion 8 and a depressible button 7 at the top. The chamber portion 8 includes a dose reservoir in fluid communication with a primary fluid reservoir in the bottle 2, for example by means of an internal tube or conduit (not shown), and optionally including valve and like flow control arrangements in familiar manner. The dose reservoir in the chamber portion is sized to hold a single dose of fluid, and the act of depressing the button 7 and allowing it to return to an undepressed position serves to prime the device by drawing such a single dose into the dose reservoir from the stock in the bottle 2 in generally familiar manner.

The unit in FIG. 1 is designed to apply fluid composition in accordance with then invention to the throat. Accordingly, the delivery system includes a deployable delivery tube 11 shown deployed horizontally for use, and which is rotatable out of a stowed configuration 11a by means of the pivoting unit 12. The delivery tube 11 comprises a hollow conduit to deliver a dose of fluid from the dose reservoir in the chamber portion via a nozzle 13 to the throat of a user. Depression of the button 7 acts in the usual way to expel the measured dose from the dose reservoir via the tube to the user. The nozzle 13 may be configured in familiar manner to create a suitable aerosolised spray of the fluid to ensure even distribution and desired spread at the application site on the user's throat.

A controlled dose is applied directly to the active site. The patient is encouraged not to swallow for, for example, 40 seconds to allow the microspheres to adhere to the mucous membranes, and thus a stabilised slow release is enabled directly at the desired active site.

The unit in FIG. 1 is a throat sprayer as an example only. It will readily be understood that the invention similarly applies to a nasal spray. Suitable modifications to the sprayer for nasal use will readily suggest themselves. In particular the nozzle is likely to point vertically, and may be incorporated as an upward extension of the button.

FIG. 2 is a SPHERULITE™ microparticle suitable for use with a composition in accordance with the invention.

Microcapsules are formed by the mixing of vegetable oils with surfactants and the glycerine-water phase under suitable shear conditions. The microcapsules (21) which are obtained by this method are multi lamellar vesicles formed with stacking of surfactant membranes. Hydrophilic components (22) are encapsulated in the aqueous phase between polar heads of tension-actives (23). Lyophilic components (24) (for example vegetable oils and Vitamin E) are encapsulated in the hydrophobic cues of tension-actives (23).

Further by way of example, three example compositions are described, the first suitable for use as a nasal spray and the second and third as a throat spray.

### Example A

Nasal Spray

| | |
|---|---|
| PURIFIED WATER (polar media; solvent) | 30-90% |
| SODIUM CHLORIDE (isotonic regulator) | 0-2.0% |
| SODIUM CARBOXYMETHYLCELLULOSE (texturant) | 0-1.0% |
| XANTHAN GUM (texturant) | 0-1.0% |
| MICROSPHERES (CMTL25) | 5-50% |
| MICROSPHERES contain: | |
| Water (solvent) | 30-90% |
| Sorbitan stearate (surfactant layer) | 1-15% |
| Polysorbate 60 (surfactant layer) | 1-15% |
| Guar hydroxypropyltrimonium chloride | 0.1-5% |
| (surfactant polymer) | |
| Calendula glycerin extract (hydrophilic; soothing) | 1-10% |
| Thymus vulgaris (hydrophobic; aroma/flavor) | 0-1.0% |
| Lavandula angustifolia (hydrophobic; aroma/flavor) | 0-1.0% |
| Menthyl lactate (hydrophobic; moisturizer/cooling) | 0-5.0% |

### Example B

Throat Spray

| | |
|---|---|
| PURIFIED WATER (polar media; solvent) | 50-90% |
| GLYCERIN (humectant) | 10-30% |
| XANTHAN GUM (texturant) | 0-1.0% |
| POTASSIUM SORBATE (preservative) | 0-1.0% |
| CITRIC ACID (acidity regulator) | 0-0.5% |
| MICROSPHERES ESAH4-50 | 5-30% |
| MICROSPHERES contain: | |
| Water (solvent) | 30-90% |
| Glycerin (hydrophilic agent; humectant) | 5-20% |
| Sorbitan stearate (surfactant layer) | 5-25% |
| Polysorbate 60 (surfactant layer) | 5-25% |
| Guar Hydroxypropyltrimonium chloride (surfactant | 0-5% |
| polymer) | |
| Olive oil (hydrophobic agent; lubricant) | 0-5% |
| Mint oil (hydrophobic agent; flavour) | 0-5% |
| Sunflower oil (hydrophobic agent; lubricant) | 0-2% |
| Vitamin E (hydrophobic agent; lubricant) | 0.1-5% |
| Potassium sorbate (preservative) | 0.05-0.5% |
| Sodium Hyaluronate powder nasal grade (hydrophilic agent; | 0.1-1.% |
| humectant) | |

### Example C

Throat Spray

| | |
|---|---|
| PURIFIED WATER (polar media; solvent) | 30-90% |
| GLYCERIN (polar media; humectant) | 5-30% |
| MICROCRYSTALLINE CELLULOSE (Texturant) | 0.1-3.0% |
| POTASSIUM SORBATE (Preservative) | 0.05-0.75% |
| SODIUM BENZOATE (Preservative) | 0.05-0.75% |
| CITRIC ACID (Acidity Regulator) | 0.05-0.5% |
| XANTHAN GUM (Texturant) | 0-0.5% |
| MICROSPHERES ESAH4-50 | 5-50% |
| MICROSPHERES contain: | |
| Purified water (solvent) | 30-90% |
| Sorbitan stearate (surfactant layer) | 5-40% |
| Polysorbate 60 (surfactant layer) | 5-40% |
| Guar Hydroxypropyltrimonium chloride | 0.1-5% |
| (surfactant polymer) | |
| Glycerin (hydrophilic agent; humectant) | 1-30% |
| Olive oil (hydrophobic agent; lubricant) | 0.1-10% |
| Peppermint oil (hydrophobic agent; aroma/flavour) | 0.1-10% |
| Grapeseed oil (hydrophobic agent; lubricant) | 0.1-10% |
| Alpha tocopherol acetate (hydrophobic agent; anti-oxidant) | 0.1-5% |
| Potassium sorbate (Preservative) | 0-0.5% |
| Citric acid (Acidity Regulator) | 0-1.0% |
| Sodium hyaluronate (hydrophilic agent; humectant) | 0-1.0% |

These compositions are examples only illustrative of but not limiting on the overall scope of the invention.

1-24. (canceled)

25. In a method for alleviating snoring by lubricating or moisturising the mucosa of the nose or throat of a human or animal subject by application thereto of at least one lubricant or moisturizer, the improvement comprising: preparing a plurality of multilamellar microparticles, the microparticles incorporating the said at least one lubricant or moisturizer, each microparticle having a positive surface charge; distributing the multilamellar microparticles in a liquid base to form a liquid composition; and providing the liquid composition for oral or nasal delivery to the said mucosa.

26. The method of claim 25, wherein the at least one lubricant or moisturizer is a natural oil.

27. The method of claim 25, wherein the microparticles additionally incorporate at least one nasal decongestant.

28. The method of claim 25, wherein the microparticles additionally incorporate at least ingredient with anti-histaminic action.

29. The method of claim 25, wherein the microparticles incorporate at least one ingredient selected from the list consisting of hyaluronic acid; sodium hyaluronate; glycerol; Calendula officinalis flower extract or glycerin extract; guar hydroxypropyltrimonium chloride; xanthan gum; cellulose gum; sodium chloride; olive oil; sunflower oil; sweet almond oil; sesame oil; Aloe vera; Aloe barbadensis; Euphorbium officinarum; oxymetazoline hydrochloride; lactoperoxidase; Thymus vulgaris; menthyl lactate; Mentha piperita; any other mint or peppermint derivative or extract; Lavendula augustifolia; a lavender derivative or extract; phenylephrine hydrochloride; pseudephedrine; ascorbic acid; acerola; Rumex crispus; eucalyptus oil; levmetamfetamine; propylhexedrine; xylometazoline hydrochloride; zincum gluconicum; menthol; eugenol; cineol; rosemary oil; summer savory oil; wild thyme oil; firtree oil; Lavendula vera oil; geranium oil; cinnamon oil; hawthorn extract; rose hips extract; cypress oil; grapeseed oil; and vitamin E.
